(19) United States
(12) Patent Application Publication
(10) Pub. No.: US 2018/0177454 A1
(43) Pub. Date: Jun. 28, 2018

(54) DEVICE AND METHOD FOR GENERATING SENSORY STIMULI FOR THE EVALUATION OF NEUROPATHY

(71) Applicant: LARA DIAGNOSTICS AB, Stockholm (SE)

(72) Inventors: Rui CHEN, Stockholm (SE); Louise WARME, Göteborg (SE)

(21) Appl. No.: 15/902,178

(22) Filed: Feb. 22, 2018

Related U.S. Application Data
(63) Continuation of application No. PCT/SE2016/050800, filed on Aug. 25, 2016.

(30) Foreign Application Priority Data
Aug. 25, 2015 (SE) 1551094-4

(51) Int. Cl. A61B 5/00

(52) U.S. Cl. CPC A61B 5/4842; A61B 5/6843; A61B 5/4029; A61B 5/0051

(57) ABSTRACT

Peripheral sensory neuropathy can be detected and quantified more accurately and objectively using a device for generating sensory stimuli in the form of vibration, comprising a vibration generator, at least one contacting element for contacting skin or tissue of a patient to transfer sensory stimuli from said vibration generator, and at least one regulating unit for regulating a parameter of said vibration, wherein the device comprises a sound generator, generating a sound which masks, attenuates or actively cancels the sound of the vibration generator. The device may further comprise a tissue support element to spatially isolate a point of contact between the tissue of the patient and the contacting element of the device, and optionally an element for controlling the pressure by which a contacting element is applied to skin or tissue of a patient. The device may also comprise at least two temperature surfaces for contacting skin or tissue of a patient and a regulating unit whereby the temperature difference between said at least two temperature surfaces can be regulated.

### TECHNICAL FIELD

The present disclosure relates to a device and method for the evaluation of neuropathy, in particular peripheral neuropathy, and especially sensory neuropathy, based on evaluation of the response of a patient to sensory stimuli.

### BACKGROUND

Neuropathy, or more accurately peripheral neuropathy, is the result of damage or disease affecting nerves in the body. The symptoms can involve impaired sensation, movement, gland or organ function, or impairment of other aspects of health, depending on the type of nerve or nerves affected. The underlying cause can be a systemic disease, such as diabetes, vitamin deficiency, medication (for example chemotherapy), traumatic injury, excessive alcohol consumption, immune system disease, or infection, or it may be genetic.

Further, peripheral neuropathy may be chronic or acute. Acute neuropathies demand urgent diagnosis, but chronic neuropathies require observation and recurrent examination to follow the progress or remission of the disease. Motor nerves, sensory nerves, or autonomic nerves may be affected. More than one type of nerve may be affected at the same time. Peripheral neuropathies may be classified according to the type of nerve predominantly involved, or by the underlying cause. Where the cause is unknown it is described as idiopathic neuropathy.

Neuropathy may cause painful cramps, fasciculation (fine muscle twitching), muscle loss, bone degeneration, and changes in the skin, hair, and nails. Neuropathy can be further divided into motor neuropathy and sensory neuropathy, exhibiting different symptoms. Motor neuropathy may cause impaired balance and coordination or, most commonly, muscle weakness. Sensory neuropathy may cause numbness to touch and vibration, reduced position sense causing poorer coordination and balance, reduced sensitivity to temperature change and pain, spontaneous tingling or burning pain, or skin allodynia (severe pain from normally non-painful stimuli, such as light touch).

This disclosure will mainly focus on sensory neuropathy, where the symptoms are very diverse, and often also highly subjective. As a consequence, both diagnosis and long-term observation of the neuropathy tends to be difficult. Different methods and devices have been developed.

The traditional method of diagnosing sensory neuropathy was to use a tuning fork to create vibrations. The tuning fork was tapped against a hard surface or the hand of the examiner to make it vibrate, and then pressed against the skin of the patient. In this fashion, the sensory nerves of, for example, the fingers and toes could be investigated by touching each finger or toe with the vibrating tune fork and asking the patient if she could feel the vibrations of the tuning fork. It is evident that such manual examination contains many sources of error, and equally evident that repeatability and sensitivity will be low. The result is likely to vary depending on how hard the tune fork is pressed against the patient's skin, the force with witch the tuning fork is tapped to make it vibrate, the area of contact with the skin etc. In most cases the tuning fork vibrates with a high intensity making it impossible to find early neuropathies, i.e. conditions where the nerves are only slightly damaged. It is also possible that the patients give an incorrect response, and imagine or pretend that they can feel the vibration already when they hear the sound of the tune fork. This can be due to auto-suggestion or a wish to appear healthier than is the case, due to shame, embarrassment, concern or a conscious or unconscious desire to keep up a healthy appearance.

In addition to testing the vibration sensitivity of the patient with the tuning fork, which tests the thick nerve fibres, thermal sensitivity is also tested. This is usually done using two glass vials or test tubes, one containing cold water and the other containing warm water. These are alternately pressed against the skin of the patient and the patient is asked which one is hot and which one is cold. Another method is to use a Y-shaped object, where one “arm” of the Y is made of a material that feels cold to the skin, e.g. metal, and the other “arm” is made of a material that feels warm, e.g. wood. It is obvious that these methods are only capable of giving a qualitative measure of the extent of neuropathy, and that no objective, quantitative measure can be obtained. This test also has low repeatability. The temperature of the water in the vials will inevitably be different at different time points, and there are no standardized materials for the hot and cold surface on the y-shaped object.

There are also other methods, for example electromyography, wherein the electrical activity in the muscles is examined in order to determine if impaired function or weakness is caused by muscle damage or nerve damage. Another method involves nerve conduction studies, in which it is assessed how the nerves and muscles of a patient respond to small electrical stimuli. In a nerve conduction study, a probe sends electrical signals to a nerve, and an electrode placed along the nerve's pathway records the nerve's response to the signals.

It is also possible to perform a nerve biopsy or a skin biopsy. In a nerve biopsy, a doctor removes a small portion of a nerve and examines it for abnormalities. In a skin biopsy, a doctor removes a small portion of skin to examine the nerve endings for signs of abnormalities. Taking a biopsy is however an invasive procedure, involving certain discomfort for the patient. Further, as the biopsy sample needs to be investigated in a laboratory, there will inevitably be a certain delay until the results are available to the physician.

As stated earlier in this background section, there are many underlying causes of neuropathy. A large patient group at risk of developing neuropathy consists of diabetics, in particular elderly suffering from diabetes. The International Diabetes Federation (IDF) have in their global guideline Managing Older People with Type 2 Diabetes, 2013, set out that “[O]lder people with diabetes should undergo examination of the peripheral nerves at the initial visit and as part of the annual review using a 10 g monofilament or 128 Hz tuning fork; a biothesiometer (cut-off point for ulcer risk >25 volts); or non-traumatic pin-prick.”

The American Diabetes Association (ADA) clinical practice recommendations contain a similar statement: “All patients with diabetes should be screened for neuropathy. After initial screening all patients should be screened annually by examining sensory function in the feet. One or more of the following tests should be used to assess sensory function: pinprick, temperature, vibration perception, pressure sensation.” It is however noted that these assessments are highly dependent on the skills of the practitioner. It is very likely that both the frequency and conduct of the test and the quality of the results widely vary in the medical community.

In an effort to develop non-invasive methods for the investigation and diagnosis of neuropathy, electronic devices comprising temperature surfaces with variable temperature, and vibration elements or surfaces, with variable vibration strength and amplitude have been developed. One experimental approach is to use the Optacon device, an electromechanical device that enables blind people to read printed material that has not been transcribed into Braille. The Optacon device consists of a main electronics unit about the size of a portable tape recorder connected by a thin cable to a camera module about the size of a penknife. The main electronics unit contains a “tactile array” onto which the blind person places his/her index finger. The Optacon user moves the camera module across a line of print, and an image of an area about the size of a letter is transmitted via the connecting cable to the main electronics unit. The tactile array in the main electronics unit contains a 24-by-6 matrix of tiny metal rods, each of which can be independently vibrated by a piezoelectric reed connected to it. Rods are vibrated that correspond to black parts of the image, thus forming a tactile image of the letter being viewed by the camera module.

When applied to the detection and/or diagnosing of sensory neuropathy, the tactile array of the Optacon is used to assess finger-tip sensation in normal and diabetic subjects. The instrument is capable of detecting the steady increase in sensory threshold with age and is able to identify peripheral neuropathy in diabetic subjects. There was little variation upon repeated testing of the same subject. (J. P. Arezzo and H. H. Schaumburg, The Use of the Optacon as a Screening Device: A New Technique for Detecting Sensory Loss in Individuals Exposed to Neurotoxins, in Journal of Occupational Medicine, Vol. 22, Issue 7, July 1980).

Another instrument used for the diagnosis and evaluation of neuropathy is the so called biothesiometer, for example the Bio-Thesiometer (supplied by the Bio-Medical Instrument Company, Ohio, USA). This is an instrument designed to measure simply and accurately the threshold of appreciation of vibration (vibration perception) in human subjects. The device is equipped with a tuning fork which works electronically and has a vibration strength that can be slowly increased until the patient can feel the vibratory sensation.

Another example is the “neuropathy diagnostic device” disclosed in U.S. Pat. No. 8,579,830, which comprises a neutral temperature surface, a variable temperature surface and a vibrating surface having variable vibration amplitude. A device closely resembling the above patent is the Dynamic Neuroscreening Device (DND, marketed by Prosenex, Hudson, N.H., USA). This is a non-invasive device used to detect peripheral neuropathy in patients with diabetes or at risk for diabetes who have yet to exhibit any clinical symptoms. The DND screens for the presence of neuropathy through the use of objective temperature and vibration sensitivity testing, using a temperature increments of 2° C. from 15 to 40° C., compared to the fixed 25° C. baseline, and using five amplitudes of 128 Hz vibration frequency.

Despite the recent developments, there remains a need for improved devices and methods for the detection and monitoring of neuropathy, in particular peripheral neuropathy, and most preferably peripheral sensory neuropathy.

### SUMMARY

One aspect of the present disclosure concerns a device for generating sensory stimuli in the form of vibration, comprising a vibration generator, at least one contacting element for contacting skin or tissue of a patient to transfer sensory stimuli from said vibration generator, and at least one regulating unit for regulating a parameter of said vibration, wherein the device further comprises a tissue support element, adapted to spatially isolate a point of contact between the tissue of the patient and the contacting element of the device, and adapted to minimize the spreading of vibration in the tissue of the patient beyond said point of contact, wherein the tissue support element comprises a pressure sensor, adapted to indicate when the pressure is within a predetermined pressure interval, and capable of giving a signal when the pressure is outside said interval. Embodiments of a device comprising a tissue support element are disclosed in the description and in the attached claims, incorporated herein by reference.

A second aspect, freely combinable with the above aspects and embodiments thereof, concerns a device which comprises a unit for controlling the pressure by which said at least one contacting element is applied to skin or tissue of a patient. Embodiments of a device comprising a pressure control element are disclosed in the description and in the attached claims, incorporated herein by reference.

A third aspect, freely combinable with the above aspects and embodiments thereof, concerns a device comprising multiple contacting elements. Preferably said multiple contacting elements can be individually activated

A fourth and further aspects concern methods of using the device according to any one of the embodiments disclosed herein, and methods of diagnosing and/or evaluating neuropathy disclosed in the description and in the attached claims, incorporated herein by reference.

The disclosure also encompasses further embodiments of said device and method, as set forth in the description and claims.

### SHORT SUMMARY OF THE DRAWINGS

The invention will be disclosed in further detail below, in the description, examples and attached drawings, in which

FIGS. 1 and 2 show a device disclosed in U.S. Pat. No. 8,579,830 representing the prior art.

FIG. 3 schematically shows a device according to different embodiments of the current description, shown in a frontal view (A), a side view (B) and a view from above (C).

FIG. 4 schematically shows an embodiment where a contacting element (31) connected to a vibration generator (33) is surrounded by a movable sheath (32) which acts as a tissue support element, and which also can be adapted to measure the pressure by which the skin and tissues of the patient is/are contacted.

FIG. 5 schematically shows another embodiment where the contacting element (31) and vibration generator (33) are flexibly arranged in the device, and the sheath (32) or tissue support element is fixed, making it possible to measure the pressure by which the skin and tissues of the patient is/are contacted by the contacting element.

FIG. 6 schematically shows an embodiment where a central processor controls (100) two temperature elements with temperature surfaces (121, 122), one vibration generator (130) with its contacting element, and a sound generator (180).

FIG. 7 schematically shows another embodiment where the set-up shown in FIG. 6 has been supplemented with a pressure control unit (135) and an optional element for communication/data transfer with external devices (190).

### DESCRIPTION

Before the present device and method is described, it is to be understood that this invention is not limited to the particular configurations, method steps, and materials disclosed herein as such configurations, steps and materials may vary somewhat. It is also to be understood that the terminology employed herein is used for the purpose of describing particular embodiments only and is not intended to be limiting since the scope of the present invention will be limited only by the appended claims and equivalents thereof.

It must also be noted that, as used in this specification and the appended claims, the singular forms “a”, “an”, and “the” include plural referents unless the context clearly dictates otherwise.

The term “about” when used in the context of numeric values denotes an interval of accuracy, familiar and acceptable to a person skilled in the art. Said interval can be ±10% or preferably ±5%.

The term “unit” is used to encompass any hardware or software implementation, or a hybrid of a hardware and software implementation. For example “a unit for measuring the contact area between a temperature surface and the skin of a patient” can be a separate device, software for operating such device, or an integrated part of the temperature surface, and software analyzing signals received from said device or integrated part.

In describing and claiming the present invention, the following terminology will be used in accordance with the definitions set out herein.

According to one aspect, this description discloses a device for generating sensory stimuli in the form of vibration, comprising a vibration generator, at least one contacting element for contacting skin or tissue of a patient to transfer sensory stimuli from said vibration generator, and at least one regulating unit for regulating a parameter of said vibration. The contact area between said contacting element and the patient is preferably at least about 5×5 mm and preferably smaller than 8×8 mm, although different size and shape of the contact area can be contemplated. Preferably the frequency of vibration is constant at about 128 Hz and the amplitude of the vibration is adjustable within an interval of about 10 μm to about 300 μm.

This device preferably comprises a sound generator, which generates a sound which masks, attenuates or actively cancels the sound of the vibration generator.

Preferably said sound generator generates a sound masking the sound of the vibration generator, wherein the sound generator is activated before the vibration generator.

According to another embodiment, the sound generator generates a sound attenuating or actively cancelling the sound of the vibration generator, wherein the sound generator is controlled by the same control unit that that controls the vibration generator in a feed-forward loop.

According to an embodiment of the above, the sound generator generates a sound attenuating or actively cancelling the sound of the vibration generator, wherein the sound generator is controlled by a control unit comprising a microphone and a processor adapted to analyze the waveform of the sound of the vibration generator, to generate an inverted sound wave directly proportional to the amplitude of the sound of the vibration generator.

Preferably the device according to any one of the embodiments set out herein also comprises a display capable of indicating the amplitude of the vibration, either in absolute numbers or expressed as a level of intensity.

Preferably the frequency of the vibrator shall be constant within ±10% during a test, preferably within ±5%, more preferably within ±2%, meaning that substantially only the amplitude shall be modulated. The vibrator preferably has a minimum vibration amplitude of 10 μm, and preferably a maximum vibration amplitude of 300 μm. Preferably it shall take less than 1 second to change the amplitude level of the vibrator from one level to any other amplitude within the above interval. Examples of levels are linearly increasing levels of amplitude, e.g. 50, 100, 150, 200, 250 and 300 μm, or levels of amplitude increasing by a multiple of two, such as 25, 50, 100, and 200 μm, or by a multiple of three, such as 10, 30, 90, and 270 μm. Further, the frequencies and amplitudes of the vibration steps shall not be affected by the true output voltage of the battery. This ensures that the test results are not affected by time since last charge.

Importantly, according to an independent embodiment, optionally combinable with any one of the above embodiments, the difference in acoustic output shall not exceed about 3 dB, preferably not more than about 1 dB, between the vibration intervals, regardless of orientation or configuration of the device. The advantage of this embodiment is that the patient should not be given any clues as to whether the vibration is increased or decreased, other than the sensory stimulation at the point of contact between the vibration element and the skin of the patient.

According to another aspect, freely combinable with the first aspects and embodiments thereof, the device further comprises a tissue support element, adapted to spatially isolate a point of contact between the tissue of the patient and the contacting element of the device, and adapted to minimize the spreading of vibration in the tissue of the patient beyond said point of contact. This addresses an important source of error, namely the spreading of vibrations in the tissue, transferring vibrations from a diseased area to a healthy area where the nerves are still functional. This also has the advantage of visually hiding the vibrating contact element, which also minimizes false responses. The tissue support element can also be part of a mechanism for measuring the pressure by which the device is held against the skin. Finally, said tissue support element can help to reduce the sound emanating from the vibration generator.

Said tissue support element is preferably spring-loaded and adapted to engage with the tissue of the patient with a predetermined pressure when the contacting element is in contact with the tissue of the patient.

More preferably said tissue support element also comprises a pressure sensor, adapted to indicate when the pressure is within a predetermined pressure interval, and capable of giving a signal when the pressure is outside said interval.

According to a third aspect, freely combinable with the first and second aspects and embodiments thereof, the device also includes a unit for controlling the pressure by which said at least one contacting element is applied to skin or tissue of a patient.

Preferably said unit for controlling the pressure by which a contacting element is applied to skin or tissue of a patient comprises a first pressure sensor associated to a first contacting element and adapted to measuring the contact pressure by which said first contacting element is placed in contact with skin or tissue of a patient.

Preferably, according to one embodiment, said first pressure sensor measures the contact pressure by measuring the pressure by which said contacting element is held against the skin or tissue of a patient.

In some embodiments, the device is adapted to measure the pressure on the vibrator and on the tissue support element, each separately. This is done primarily in order to ensure that both the vibrator and the tissue support element is in contact with the patient, and not only one of them. In some embodiments, both these measurements may be done by said first pressure sensor. In some embodiments, the first pressure sensor is adapted to measure the pressure of the tissue support element and a second sensor is adapted to measure the pressure of the vibrator.

Furthermore, the device may be adapted to ensure that the tissue support element is in contact with the patient by using displacement comparisons. The tissue support element has an original position and the vibrator has an original position. By measuring their positions and comparing them with their original position, it is possible to measure the displacement of the tissue support element and the vibrator, both relative to their respective original positions and relative to on another.

Preferably, but according to another embodiment, said first pressure sensor measures the contact pressure by measuring the attenuation of the vibration of said contact element.

Preferably said vibration generator is activated only when a predetermined minimum contact pressure is reached. Similarly, said vibration generator is preferably deactivated when a predetermined maximum contact pressure is reached.

According to a fourth aspect, freely combinable with the previous aspects and embodiments thereof, the device comprises multiple contacting elements. Preferably said multiple contacting elements can be individually activated.

According to a fifth aspect, freely combinable with the previous aspects and embodiments thereof, the device comprises at least two temperature surfaces for contacting skin or tissue of a patient and a regulating unit whereby the temperature difference between said at least two temperature surfaces can be regulated. Preferably said at least two temperature surfaces are oblique, or in other words positioned at an angle in relation to each other.

According to an embodiment, at least one temperature surface is associated to a temperature element whereby the temperature of said surface can be individually adjusted with high accuracy. This makes it possible to create a temperature difference between said temperature surfaces, which enables temperature sensitivity testing using the forced-choice method.

According to an aspect of said embodiment, both temperature surfaces are associated to individual temperature elements whereby the temperature of each surface can be adjusted to a different temperature, creating a temperature difference between said temperature surfaces. At least one temperature surface, preferably both temperature surfaces, includes an element for measuring the temperature of said surface.

According to an aspect of the above embodiments, the device further comprises a display where the temperature of the temperature surfaces, and/or the difference between said at least two temperature surfaces can be indicated. Preferably the display is also capable of indicating the current vibration level, e.g. the intensity and/or amplitude of the vibrator.

According to an aspect freely combinable with the above aspects and embodiments, said temperature element (-s) is (are) chosen from thermoelectric elements, resistive heating elements (metallic, ceramic or composite), refrigerant based elements, and elements based on chemical reactions (endothermic and exothermic reactions).

The temperature difference between the temperature elements should be freely adjustable in the interval of about 10 to about 45° C., preferably 10-45° C. and possible to control with an accuracy of 2° C. This means that when the “cold” temperature element is maintained at a temperature of for example 10° C., the “warm” temperature element can be set to a temperature of 12° C., and incrementally increased, for example to 14° C., 16° C., and so on. According to an embodiment, it is possible to rapidly change the temperature of the elements. Preferably it should take less than 10 seconds to change the temperature from one temperature to another within the above specified range. Further, the temperature of a temperature element may never exceed 48° C., nor should the temperature of a temperature element ever be lower than 0° C. at normal operating conditions.

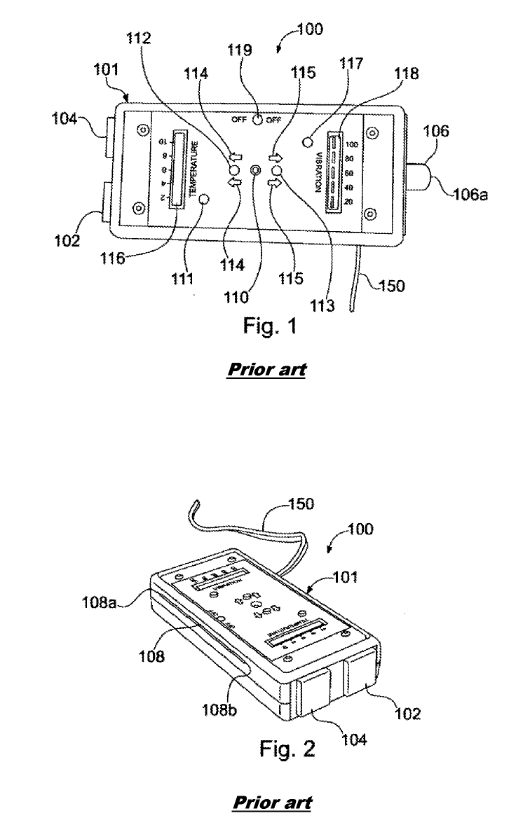

FIGS. 1 and 2 show a device representing the prior art, having both temperature surfaces and a vibrating element for contacting the skin or tissue of a patient.

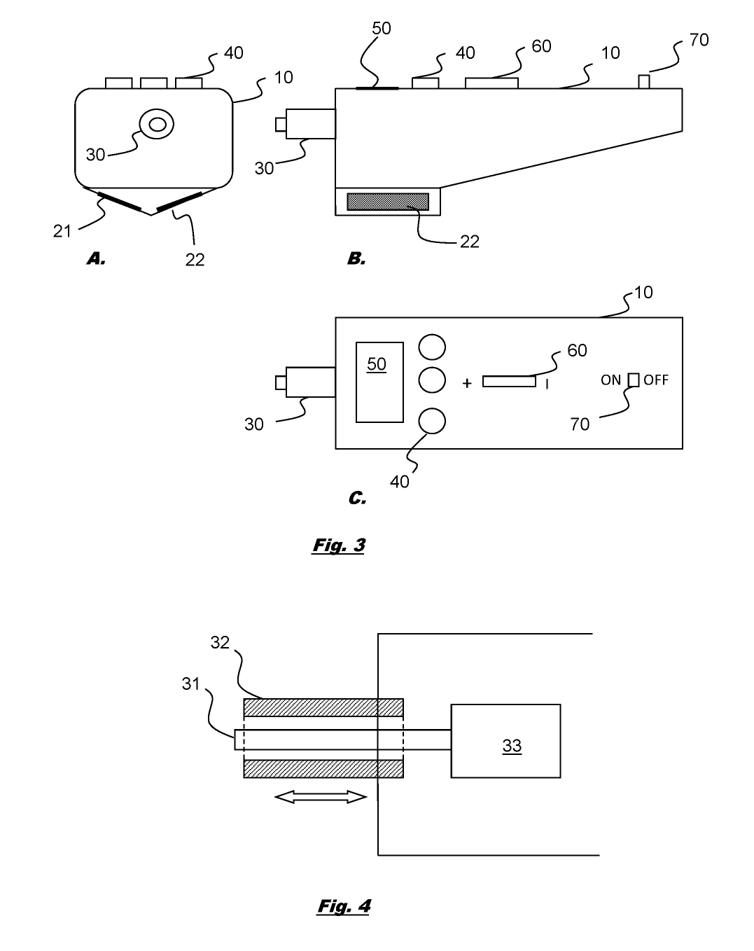

FIG. 3 shows an improved device incorporating one or more of the aspects and embodiments set out herein. FIG. 3 is schematic, and shows in panel A) a frontal view of a device 10 having two temperature surfaces 21 and 22 positioned at an angle to each other. A vibrating contact element 30 is also shown, as well as controls 40 for selecting the mode of operation. The device further has a display 50, and a control 60 for increasing or reducing the amplitude of vibration, when the device is in “vibration mode” and increasing or reducing the temperature of at least one temperature surface, or increasing or reducing the temperature difference between at least two temperature surfaces, when the device is in “temperature mode”. The device further has a control 70 for activating and deactivating the device. Panel B) shows a side view, and panel C) a top view. The drawing is only schematic, and does not indicate the size and proportions of the device. The device is however preferably hand held, which places natural limitations to the length, width and weight of the device. The device may further include a power inlet for charging batteries in the device, or for powering the device in absence of batteries. The device may also have a port for communication, for example a USB port, an IR port, Bluetooth, or the like. The device may also have a clip for attachment to a belt or pocket.

FIG. 4 schematically shows a detail of an embodiment, where a vibrating contact element 31 is surrounded by a tissue support element 32. The vibrating contact element is connected to a vibration generator 33. The tissue support element 32 is preferably flexible, either in itself, or by being movably connected to the device. This has many advantages, as the element 32 helps to support the tissue, preventing the vibrations to spread in the tissue of the patient. The element also helps to “hide” the movement of the contacting element, reducing the risk for false responses by the patient. Also, the element 32 can constitute part of a unit for measuring the contact pressure.

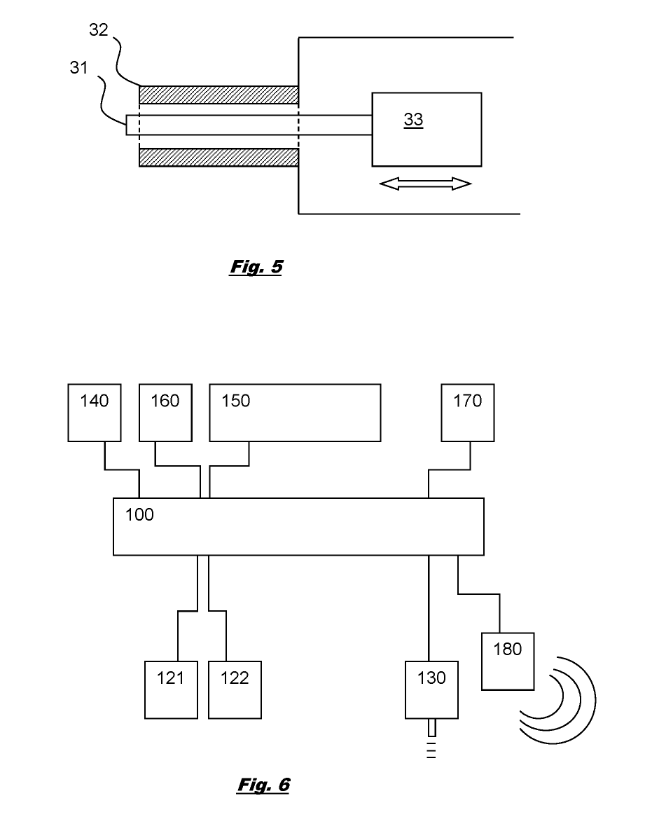

FIG. 5 shows another embodiment where the tissue support element is fixed to the device, and the contacting element 31 is movable. This makes it possible to measure the pressure by which the contacting element is held towards the skin of a patient. At the same time, the element 32 can have the same functions as outlined above.

FIG. 6 shows a schematic view of a device wherein a central processor 100 is connected to a display 150, to temperature surfaces 121 and 122, a vibrator 130 and to controls 140, 160 and 170. The controls can be for examples controls 170 for activating/deactivating the device, for example ON/OFF, or ON/OFF/STANDBY. According to one embodiment, again freely combinable with other embodiments presented herein, there are also controls 140 for selecting the mode of operation, e.g. “vibration test” or “temperature test” and a control 160 for increasing or reducing the output in each mode. For example, in “vibration test” mode, an increase means an increase of the amplitude of vibration. Conversely, in “temperature test” mode, an increase or decrease means an increase or decrease of the temperature difference, or an increase or decrease of the temperature of an individual temperature surface. Importantly, the device also comprises a sound generator 180 which masks, attenuates or camouflages the sound of the vibration element/vibration generator.

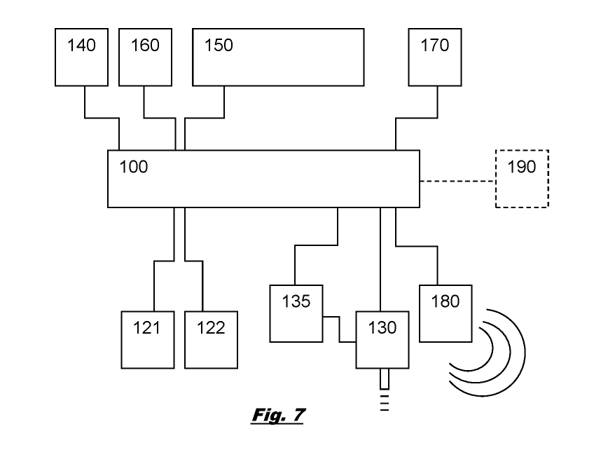

FIG. 7 shows a schematic view of a device wherein a central processor 100 is connected to a display 150, to temperature surfaces 121 and 122, a vibrator 130 and to controls 140, 160 and 170. The device also comprises a unit 135 for controlling the pressure of the contacting element against the skin of the patient. The device also optionally comprises a communications port 190 such as an USB-port, transmitter/receiver of radio signals, IR, Bluetooth etc.

A device as disclosed herein can be used in the following fashion: The device is activated by turning on the power. The operator, a nurse or physician, selects the mode of operation. In the “vibration test” mode, the intensity of vibration is set to the lowest level, and the contact element brought in contact with the skin of a patient. The physician touches in turn each toe or finger with the vibrating contact element. The amplitude of vibration is increased, either gradually or incrementally, until the patient senses the vibration. The level of vibration discernible to the patient is recorded as a measure of the patient's condition. In severe cases of neuropathy, the patient is possibly unable to sense even strong vibration, whereas in milder cases, the patient is still able to discern low or medium intense vibration.

In “temperature test” mode, the operator sets the temperature of one temperature surface at a first temperature, preferably a temperature at the lower end of the temperature interval, e.g. 10° C., alternatively starts at ambient temperature. The temperature sensitivity of a patient can be tested using one temperature surface, but is preferably tested using two surfaces, alternatingly brought in contact with the skin of the patient. The patient is requested to indicate which of the two surfaces is colder or warmer. The operator gradually increases the temperature difference between the two surfaces, until the patient can indicate correctly which surface is colder or warmer, respectively. The temperature difference, as well as the absolute temperatures, is then recorded.

In a general embodiment, this specification also discloses a method of diagnosing and/or evaluating the progress of neuropathy, wherein a device according to any one of the above claims is used.

More specifically, according to an embodiment, a method of diagnosing and/or evaluating the progress of neuropathy is disclosed, wherein a patient is subjected to stimuli in the form of vibration, directed to a specific location of the body of said patient, using a vibration generator and a contact element, wherein a sound masking, attenuating or actively canceling the sound of the vibration is emitted simultaneously.

According to another embodiment, a method of diagnosing and/or evaluating the progress of neuropathy also involves preventing the stimuli in the form of vibration from spreading in the skin or tissue of the patient by applying a tissue support element to the skin of the patient.

According to another embodiment, a method involves steps or means, preventing the patient from seeing the vibrating contact element when this is applied to the skin of the patient.

According to another embodiment, the pressure by which the contacting element is applied to the skin of the patient is measured, and a different indication is given when the pressure is below, at or above a pre-determined pressure. According to a non-limiting example, a traffic light principle could be applied, where an insufficient contact pressure could be indicated with a yellow light, an excessive pressure indicated with a red light, and the desired, pre-determined pressure indicated with a green light. Other types of feed back signals are also contemplated, including different graphic symbols and animations.

According to another embodiment, the pressure by which the contacting element is applied to the skin of the patient is measured, and the vibration generator is activated only when the pressure is within a pre-determined pressure interval.

The methods above are applicable to different investigations and diagnosis. Preferably, in any one of the embodiments of the method, the neuropathy is peripheral sensory neuropathy.

According to another embodiment, the device comprises a sound generator, which generates a sound which masks, attenuates or actively cancels the sound of the vibration generator.

According to another embodiment, the device comprises at least two temperature surfaces for contacting skin or tissue of a patient and a regulating unit whereby the temperature difference between said at least two temperature surfaces can be regulated.

Although the invention has been described with regard to its preferred embodiments, which constitute the best mode presently known to the inventors, it should be understood that various changes and modifications as would be obvious to one having the ordinary skill in this art may be made without departing from the scope of the invention which is set forth in the claims appended hereto.

1. A device for generating sensory stimuli in the form of vibration, comprising a vibration generator, at least one contacting element for contacting skin or tissue of a patient to transfer sensory stimuli from said vibration generator, and at least one regulating unit for regulating a parameter of said vibration, wherein the device comprises a tissue support element, adapted to spatially isolate a point of contact between the tissue of the patient and the contacting element of the device, and adapted to minimize the spreading of vibration in the tissue of the patient beyond said point of contact, wherein the tissue support element comprises a pressure sensor, adapted to indicate when the pressure is within a predetermined pressure interval, and capable of giving a signal when the pressure is outside said interval.

2. The device according to claim 1, wherein said tissue support element is spring-loaded and adapted to engaging with the tissue of the patient with a predetermined pressure when the contacting element is in contact with the tissue of the patient.

3. The device according to claim 1, wherein the device further comprises a unit for controlling the pressure by which said at least one contacting element is applied to skin or tissue of a patient.

4. The device according to claim 3, wherein said unit for controlling the pressure by which a contacting element is applied to skin or tissue of a patient comprises a first pressure sensor associated to a first contacting element and adapted to measuring the contact pressure by which said first contacting element is placed in contact with skin or tissue of a patient.

5. The device according to claim 4, wherein said first pressure sensor measures the contact pressure by measuring the pressure by which said contacting element is held against the skin or tissue of a patient.

6. The device according to claim 4, wherein said first pressure sensor measures the contact pressure by measuring the attenuation of the vibration of said contact element.

7. The device according to claim 3, wherein said vibration generator is activated only when a predetermined minimum contact pressure is reached.

8. The device according to claim 3, wherein said vibration generator is deactivated when a predetermined maximum contact pressure is reached.

9. The device according to claim 1, wherein said device comprises multiple contacting elements.

10. The device according to claim 9, wherein said multiple contacting elements can be individually activated.

11. A method of diagnosing and/or evaluating the progress of neuropathy, wherein a device according to claim 1 is used.

12. A method of diagnosing and/or evaluating the progress of neuropathy, wherein a patient is subjected to stimuli in the form of vibration, directed to a specific location of the body of said patient, using a vibration generator and a contact element, wherein the stimuli in the form of vibration is prevented from spreading in the skin or tissue of the patient by applying a tissue support element to the skin of the patient.

13. The method according to claim 12, wherein the patient is prevented from seeing the vibrating contact element when this is applied to the skin of the patient.

14. The method according to claim 12, wherein the pressure by which the contacting element is applied to the skin of the patient is measured, and wherein a different indication is given when the pressure is below, at or above a pre-determined pressure.

15. The method according to claim 12, wherein the pressure by which the contacting element is applied to the skin of the patient is measured, and wherein the vibration generator is activated only when the pressure is within a pre-determined pressure interval.
